SEQUENCE LISTING (1 sequence)

SEQ ID NO: 1 - protein, 275 aa, Artificial Sequence
  Synthetic Construct
HGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2023/0088005 A1
(43) Pub. Date: Mar. 23, 2023

(54) THERAPEUTIC USES OF DULAGLUTIDE

(71) Applicant: Eli Lilly and Company, Indianapolis, IN (US)

(72) Inventor: Hertzel Chaim GERSTEIN, Toronto (CA)

(21) Appl. No.: 17/795,797

(22) PCT Filed: Jan. 22, 2021
(86) PCT No.: PCT/US2021/014525

§ 371 Date: Jul. 27, 2022

Related U.S. Application Data
(60) Provisional application No. 62/967,790, filed on Jan. 30, 2020.

(51) Int. Cl. A61K 38/26; A61P 25/00; A61P 3/10

(52) U.S. Cl. CPC A61K 38/26; A61P 25/00; A61P 3/10

(57) ABSTRACT

The present invention relates to methods for treating, preventing or delaying disorders relating to cognition, such as cognitive decline, cognitive impairment or dementia in a patient. Specifically, the method comprising administering dulaglutide in a therapeutically effective amount to the patient once weekly. Further disclosed are methods for improving glycemic control in a patient with type 2 diabetes mellitus, comprising administering dulaglutide in a therapeutically effective amount for reducing the risk of the patient experiencing cognitive decline.

The present invention relates to the field of medicine. More particularly, the present invention relates to methods for treating, preventing or delaying disorders relating to cognition, such as cognitive decline, cognitive impairment or dementia.

Patients with type 2 diabetes mellitis (T2DM) frequently suffer from a variety of comorbidities, including cognitive issues such as cognitive decline, cognitive impairment or dementia. People with diabetes are 1.5 to 2 times more likely than unaffected people to experience cognitive decline, minimal cognitive impairment or dementia. This relationship is independent of other risk factors for cognitive dysfunction and accounts for a prevalence of 13% in people with diabetes aged 65-74 years and 24% in people aged 75 years or older. No single cause has been identified for the high risk of cognitive dysfunction in people with diabetes. The possibility that certain diabetes treatments, including glucagon-like peptide-1 (GLP-1) receptor agonists, may have benefits on cognitive function has been proposed and is being studied. See, e.g., GD Femminella, et al., Evaluating the effects of the novel GIP-1 analogue liraglutide in Alzheimer's disease: study protocol for a randomized controlled trial (ELAD study), 20 TRIALS 191 (2019). There remains a need, however, for therapies to treat, prevent or delay cognitive decline, cognitive impairment or dementia, including in patients with T2DM.

Accordingly, the present invention provides a method of treating, preventing or delaying development of a cognitive disorder in a patient, comprising administering dulaglutide in a therapeutically effective amount to the patient once weekly.

In another aspect, the present invention provides a method of preventing or delaying cognitive decline in a patient, comprising administering dulaglutide in a therapeutically effective amount to the patient once weekly.

In another aspect, the present invention provides a method of improving glycemic control and treating, preventing or delaying cognitive decline in a patient in a patient with type 2 diabetes mellitus, comprising administering dulaglutide in a therapeutically effective amount to the patient once weekly.

In another aspect, the present invention provides a method of improving glycemic control in a patient with type 2 diabetes mellitus, comprising administering dulaglutide in a therapeutically effective amount to the patient once weekly, wherein the method results in a reduction in the risk of the patient experiencing cognitive decline.

In another aspect, the present invention provides dulaglutide for use in treating, preventing or delaying development of a cognitive disorder in a patient, comprising administering dulaglutide in a therapeutically effective amount to the patient once weekly.

In another aspect, the present invention provides use of dulaglutide for the preparation of a medicament for treating, preventing or delaying development of a cognitive disorder in a patient, comprising administering dulaglutide in a therapeutically effective amount to the patient once weekly.

Dulaglutide is a human GLP-1 receptor agonist which comprises a dimer of a GLP-1 analog fused at its C-terminus via a peptide linker to the N-terminus of an analog of an Fc portion of an immunoglobulin, and is identified by CAS registry number 923950-08-7, which provides the following chemical name: 7-37-Glucagon-like peptide I [8-glycine, 22-glutamic acid, 36-glycine] (synthetic human) fusion protein with peptide (synthetic 16-amino acid linker) fusion protein with immunoglobulin G4 (synthetic human Fc fragment), dimer. Each monomer of dulaglutide has the amino acid sequence set forth in SEQ ID NO:1:

(SEQ ID NO: 1)
HGEGTFTSDVSSYLEEQAAKEFIAWLVKGG
GGGGGSGGGGSGGGGSAESKYGPPCPPCPA
PEAAGGPSVFLFPPKPKDTLMISRTPEVTC
VVVDVSQEDPEVQFNWYVDGVEVHNAKTKP
REEQFNSTYRVVSVLTVLHQDWLNGKEYKC
KVSNKGLPSSIEKTISKAKGQPREPQVYTL
PPSQEEMTKNQVSLTCLVKGFYPSDIAVEW
ESNGQPENNYKTTPPVLDSDGSFFLYSRLT
VDKSRWQEGNVFSCSVMHEALHNHYTQKSL
SLSLG.

The two monomers are attached by disulfide bonds between the cysteine residues at positions 55 and 58 of SEQ ID NO:1 to form the dimer. Dulaglutide's structure, function, production and use in treating T2DM is described in more detail in U.S. Pat. No. 7,452,966 and U.S. Patent Application Publication No. US20100196405. When used herein, the term “dulaglutide” refers to any GLP-1 receptor agonist protein dimer of two monomers having the amino acid sequence of SEQ ID NO:1, including any protein that is the subject of a regulatory submission seeking approval of a GLP-1 receptor agonist product which relies in whole or part upon data submitted to a regulatory agency by Eli Lilly and Company relating to dulaglutide, regardless of whether the party seeking approval of said protein actually identifies the protein as dulaglutide or uses some other term. Dulaglutide agonizes the GLP-1 receptor resulting in stimulation of insulin synthesis and secretion, and has been shown to provide improved glycemic control in T2DM patients.

The present application is directed to the recent discovery that dulaglutide is capable of delaying or preventing cognitive decline.

The development or progression of cognitive decline may be reflected in scores generated through administration of measures of cognitive status, such as the Montreal Cognitive Assessment (MoCA) and the Digit Symbol Substitution Test (DSST).

The MoCA is a cognitive screening test that has been validated in the setting of mild cognitive impairment and subsequently adopted in numerous clinical settings. The test comprises a 1-page 30-item questionnaire designed to be administered in approximately 10 minutes in the participant's first language using a validated translation. It assesses seven cognitive domains including short-term memory, visuospatial abilities, executive function, attention, concentration, working memory and language. See, e.g., Nasreddine Z S, et al. The Montreal Cognitive Assessment, MoCA: a brief screening tool for mild cognitive impairment. J. AM. GERIATR. SOC. 2005; 53(4): 695-9; Pendlebury S T, et al., Underestimation of cognitive impairment by Mini Mental State Examination versus the Montreal Cognitive Assessment in patients with transient ischemic attack and stroke: a population-based study. STROKE 2010; 41(6): 1290-3. The MoCA score is equal to the number of items out of 30 answered correctly, plus 1 extra point if the individual reports≤12 years of education. Controlled validation studies in cognitively normal individuals have reported a mean MoCA score of 27.4 with a standard deviation of 2.2. However, population-based studies of presumably cognitively intact people report much lower mean scores. See, e.g., Rossetti H C, et al., Normative data for the Montreal Cognitive Assessment (MoCA) in a population-based sample. NEUROLOGY 2011; 77(13): 1272-5.

The DSST is a subtest of the Wechsler Adult Intelligence Scale (3rd Edition) and assesses a wide array of cognitive domains including visual-motor speed and coordination, capacity for learning, attention, concentration and short-term memory. See Wechsler D, Manual for the Wechsler Adult Intelligence scale. NY, N.Y. (1955); D W. The Wechsler Adult Intelligence Scale-Revised. N.Y., NY: The Psychological Corporation (1981). It consists of rows of 9 randomly ordered symbols with a blank square underneath, and a key at the top of the page that pairs each symbol to a number. Respondents fill the blank space under each symbol with the corresponding number as quickly as possible over a 2-minute period. The score is the number of consecutive digit-symbol pairs correctly completed within 2 minutes, and the maximum possible score is 135. It has been extensively used to measure cognitive function in cognitively intact individuals with and without diabetes, and validation studies have demonstrated that it predicts future cognitive dysfunction and disability; is relatively easy to administer; is not language specific; and its score is correlated with measures of physical function and future cognitive decline. See, e.g., Rosano C, et al., Association Between Lower Digit Symbol Substitution Test Score and Slower Gait and Greater Risk of Mortality and of Developing Incident Disability in Well-Functioning Older Adults, J. AM. GERIATR. SOC. (2008); Knopman D, et al., Cardiovascular risk factors and cognitive decline in middle-aged adults. NEUROLOGY. 56(1):42-8 (2001). When the DSST was used in randomized controlled trials involving cognitively intact people with dysglycemia, mean baseline scores ranged from 36 to 52. See Cukierman-Yaffe T, et al., Effects of basal insulin glargine and omega-3 fatty acid on cognitive decline and probable cognitive impairment in people with dysglycaemia: a substudy of the ORIGIN trial. THE LANCET DIABETES & ENDOCRINOLOGY 2014; 2(7): 562-72; Launer L J, et al. Effects of intensive glucose lowering on brain structure and function in people with type 2 diabetes (ACCORD MIND): a randomised open-label substudy. LANCET NEUROL. (2011); 10(11): 969-77.

Previous studies have suggested that the normal range of cognitive tests scores may differ by country. Rossetti H C, et al., Normative data for the Montreal Cognitive Assessment (MoCA) in a population-based sample. NEUROLOGY 2011; 77(13): 1272-5; Thomann A E, et al. The Montreal Cognitive Assessment: Normative Data from a German-Speaking Cohort and Comparison with International Normative Samples. J. ALZHEIMER'S DIS. 2018; 64(2): 643-55. Thus, when assessing substantive decline in populations that include individuals from multiple countries, it may be desirable to standardize each individual's score at each time-point to a mean baseline score and standard deviation of the values achieved within his or her country, as opposed to the mean and standard deviation of the entire population.

Previous studies have also noted that cognitive test scores decline very slowly with time in groups of middle-aged individuals, but that some individuals experience faster decline than others. See Cukierman-Yaffe T, et al. Effects of basal insulin glargine and omega-3 fatty acid on cognitive decline and probable cognitive impairment in people with dysglycaemia: a substudy of the ORIGIN trial. THE LANCET DIABETES & ENDOCRINOLOGY 2014; 2(7): 562-72; Proust-Lima C, et al., Sensitivity of four psychometric tests to measure cognitive changes in brain aging-population-based studies. Am. J. EPIDEMIOL. 2007; 165(3): 344-50. These and other studies support the use of a threshold-based definition of decline by 1.5 standard deviations or greater from baseline. See also Group S R, et al. Effect of Intensive vs Standard Blood Pressure Control on Probable Dementia: A Randomized Clinical Trial. JAMA 2019; 321(6): 553-61; Group S R. A Randomized Trial of Intensive versus Standard Blood-Pressure Control. N. ENGL. J. MED. 2015; Biessels G J, et al. Rationale and design of the CAROLINA(R)—cognition substudy: a randomised controlled trial on cognitive outcomes of linagliptin versus glimepiride in patients with type 2 diabetes mellitus. BMC NEUROL. 2018; 18(1): 7.

When used herein, the term “cognitive disorder” refers to any condition involving impairments in a person's cognitive function, such as difficulties with memory, learning new things, ability to concentrate and/or decision-making that affects the person's everyday life. Such impairment ranges from mild cognitive impairment (MCI) to mild, moderate and severe dementia. MCI refers to a stage of cognitive impairment between the expected cognitive changes consistent with aging and mild dementia, and may be characterized by a slight but noticeable and measurable decline in cognitive abilities, including memory and thinking skills, but without loss in ability to undertake everyday activities. More severe impairment, or dementia, is associated with losses in ability to perform everyday activities, and depending on the severity, the abilities to read, write, and/or understand meaning or significance of things.

When used herein, the term “substantive cognitive decline” or “SCD” refers to a significant decrease in a subject's score in a standardized cognitive assessment, such as MoCA or DSST of 1.5 standard deviations or greater.

When used herein, the terms “country-standardization,” “country-standardized,” and the like, refer to normalization of cognitive function scores by: calculating the baseline mean and standard deviation of the scores within each country; and using these baseline mean and standard deviations to calculate a standardized MoCA and DSST score for each participant at each time point by subtracting the country-specific baseline mean score from each individual's score at that time point and dividing the difference by the country-specific baseline standard deviation.

The methods provided herein may be most effective in patients at relatively higher risk for experiencing cognitive decline. In certain embodiments, such patients are those having one or more of: T2DM; hypertension; elevated cholesterol and/or obesity.

In certain embodiments, such patients have established cardiovascular disease; and/or one or more risk factors for major adverse cardiovascular events.

When used herein, the term “major adverse cardiovascular events” refers to cardiovascular death, non-fatal myocardial infarction and non-fatal stroke. These events are also sometimes referred to as MACE or MACE 3 events. The first to occur of any of these events is a composite endpoint frequently used in CVOTs.

When used herein in relation to major adverse cardiovascular events, the term “risk factors” refers to characteristics of T2DM patients understood to increase their risk for a major adverse cardiovascular event. Such risk factors include in particular any of the following: current tobacco use (any form of tobacco); use of at least 1 approved lipid modifying therapy (e.g., statins such as atorvastatin, rosuvastatin, simvastatin, pravastatin, lovastatin, fluvastatin or pitavastatin; PCSK9 inhibitors, such as evolocumab or alirocumab; and ezetimibe) to treat hypercholesterolemia or a documented untreated low-density lipoprotein cholesterol (LDL-C)≥3.4 mmol/L (130 mg/dL) within the past 6 months; documented treated or untreated high-density lipoprotein cholesterol (HDL-C)<1.0 mmol/L (40 mg/dL) for men and <1.3 mmol/L (50 mg/dL) for women or triglycerides≥2.3 mmol/L (200 mg/dL) within the past 6 months; use of at least 1 blood pressure medication to treat hypertension (e.g., angiotensin converting enzyme (ACE) inhibitors, angiotensin receptor blockers (ARBs), thiazidelike diuretics, and dihydropyridine calcium channel blockers) or untreated systolic blood pressure (SBP)≥140 mm Hg or diastolic blood pressure (DBP)≥95 mmHg; measured waist-to-hip ratio>1.0 for men and >0.8 for women.

When used herein, the terms “treatment,” “treat,” “treating,” and the like, are meant to include slowing or attenuating the progression of a disease, condition or disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed. When used herein, the terms “prevent,” “preventing,” “prevention,” and the like, are meant to include avoidance of the onset of a disease, condition, disorder or symptom. When used herein, the terms “delay,” “delaying,” and the like, are meant to include increasing the duration of time that occurs until onset of a disease, condition, disorder or symptom.

When used herein in connection with multiple outcomes, the term “composite” refers to the first to occur of any of the outcomes.

When used herein, the term “hazard ratio” refers to a measure of the relative rate of progression to an endpoint as compared to a control group. In outcome-based clinical trials, a reduction in the hazard ratio for a test arm as compared to the control indicates the therapy used in the test arm reduces the risk of the endpoint, in the case of the studies described herein, major adverse cardiovascular events.

“Therapeutically effective amount” means the amount of dulaglutide for the methods and uses of the present invention or pharmaceutical composition comprising dulaglutide for the methods and uses of the present invention that will elicit the biological or medical response of or desired therapeutic effect on the patient that is being sought by the researcher, medical doctor, or other clinician. An effective amount of dulaglutide may vary according to factors such as the disease state, age, sex, and weight of the individual, and the ability of dulaglutide to elicit a desired response in the individual. An effective amount is also one in which any toxic or detrimental effect is outweighed by the therapeutically beneficial effects. In certain embodiments, the therapeutically effective amount of dulaglutide for use in the methods described herein is selected from the group consisting of 1.5, 3.0 and 4.5 mg. In certain embodiments, the therapeutically effective amount of dulaglutide is 3.0 mg. In certain embodiments, the therapeutically effective amount of dulaglutide is 4.5 mg. In preferred embodiments, the therapeutically effective amount of dulaglutide is 1.5 mg.

Additional embodiments of the present invention are described below:

A method of treating, preventing or delaying development of a cognitive disorder in a patient, comprising administering dulaglutide in a therapeutically effective amount to the patient once weekly. In an embodiment, the cognitive disorder is selected from the group consisting of MCI and dementia.

A method of preventing or delaying cognitive decline in a patient, comprising administering dulaglutide in a therapeutically effective amount to the patient once weekly.

A method of improving glycemic control and treating, preventing or delaying cognitive decline in a patient in a patient with type 2 diabetes mellitus, comprising administering dulaglutide in a therapeutically effective amount to the patient once weekly.

In an embodiment, the method results in a reduction in the risk of the patient experiencing cognitive decline.

A method of improving glycemic control in a patient with type 2 diabetes mellitus, comprising administering dulaglutide in a therapeutically effective amount to the patient once weekly, wherein the method results in a reduction in the risk of the patient experiencing cognitive decline.

The method of any of the above embodiments wherein the patient has type 2 diabetes mellitus.

The method of any of the above embodiments wherein the patient has one or more of: T2DM; hypertension; elevated cholesterol and obesity.

In an embodiment, the patient has either: multiple cardiovascular risk factors without established cardiovascular disease; or established cardiovascular disease.

In an embodiment, the risk factors for cardiovascular disease are selected from the group consisting of: current tobacco use (any form of tobacco); use of at least 1 approved lipid modifying therapy to treat hypercholesterolemia or a documented untreated low-density lipoprotein cholesterol (LDL-C)≥3.4 mmol/L (130 mg/dL) within the past 6 months; documented treated or untreated high-density lipoprotein cholesterol (HDL-C)<1.0 mmol/L (40 mg/dL) for men and <1.3 mmol/L (50 mg/dL) for women or triglycerides≥2.3 mmol/L (200 mg/dL) within the past 6 months; use of at least 1 blood pressure medication to treat hypertension or untreated systolic blood pressure (SBP)≥140 mm Hg or diastolic blood pressure (DBP)≥95 mmHg; measured waist-to-hip ratio>1.0 for men and >0.8 for women.

In an embodiment, the cognitive disorder is selected from the group consisting of MCI and dementia.

In an embodiment, the patient's risk of cognitive decline is reduced by at least about 14%.

In an embodiment, the risk of a major adverse cardiovascular event is reduced by at least about 10%.

In an embodiment, the risk of a major adverse cardiovascular event is reduced by at least about 11%.

In an embodiment, the risk of a major adverse cardiovascular event is reduced by about 12%.

In an embodiment, the risk of the occurrence of a composite of the following outcomes is reduced: cognitive decline or death.

In an embodiment, the risk of the occurrence of a composite of the following outcomes is reduced: cognitive decline or stroke.

In an embodiment, the risk of the occurrence of a composite of the following outcomes is reduced: cognitive decline, stroke or transient ischemic attack.

In an embodiment, the risk of the occurrence of a composite of the following outcomes is reduced: cognitive decline, stroke, transient ischemic attack or death.

In an embodiment, the risk of cardiovascular death is lower.

In an embodiment, the risk of non-fatal stroke is lower.

In an embodiment, the risk of non-fatal myocardial infarction is lower.

In an embodiment, the risk of the occurrence of a composite of the following outcomes is reduced: diabetic retinopathy needing laser, anti-VEGF therapy, or vitrectomy; clinical proteinuria; a 30% decline in eGFR; or chronic renal replacement therapy.

In an embodiment, the amount of dulaglutide is selected from the group consisting of about 1.5 mg, about 3.0 mg and about 4.5 mg.

In an embodiment, the amount of dulaglutide is about 1.5 mg.

In an embodiment, the amount of dulaglutide is about 3.0 mg.

In an embodiment, the amount of dulaglutide is about 4.5 mg.

In an embodiment, once weekly administration of dulaglutide is continued for at least 2 years.

In an embodiment, once weekly administration of dulaglutide is continued for at least 3 years.

In an embodiment, once weekly administration of dulaglutide is continued for at least 4 years.

In an embodiment, once weekly administration of dulaglutide is continued for approximately 5 years.

In an embodiment, once weekly administration of dulaglutide is continued for at least 5.4 years.

In an embodiment, the patient is also administered the standard of care for reducing the risk of major adverse cardiovascular events.

In an embodiment, the patient is also administered the maximutn tolerated dose of an ACE inhibitor.

In an embodiment, the patient is also administered the maximum tolerated dose of an ARB.

In an embodiment, the patient is also administered a beta blocker.

In an embodiment, the patient is also administered a calcium channel blocker.

In an embodiment, the patient is also administered a diuretic.

In an embodiment, the patient is also administered an antithrombotic agent.

In an embodiment, the patient is also administered aspirin.

In an embodiment, the patient is also administered a statin.

Dulaglutide for use in any of the above embodiments.

Use of dulaglutide in the preparation of a medicament for any of the above embodiments.

Further embodiments are described in the examples below, which are not to be construed as limiting.

### EXAMPLES

A phase 3 clinical study named Researching Cardiovascular Events with a Weekly INcretin in Diabetes (REWIND) is designed to assess the effect of once-weekly administration of dulaglutide compared to placebo on major adverse CV events when added to the existing antihyperglycemic regimen of patients with type 2 diabetes who are at high risk for CV events. The enrollment criteria, set forth in Table 1 below, are designed to include participants who are similar to patients seen within a typical diabetes practice, who have varying cardiovascular risk factors or established cardiovascular disease:

TABLE 1
Enrollment Criteria.
Key inclusion criteria

T2DM with HbA1c ≤9.5%
Stable dose of 0, 1 or 2 oral glucose-lowering drugs ± basal insulin for ≥3 months
BMI ≥23 kg/m²
If age ≥50 years, at least 1 of: prior MI; prior ischaemic stroke; coronary
revascularization ≥2 years earlier; carotid or peripheral revascularization ≥2 months
earlier; unstable angina hospitalization; image proven myocardial ischaemia; or
percutaneous coronary intervention
If age ≥55 years, any of the above or at least 1 of: documented myocardial ischaemia
by stress test or imaging; >50% coronary, carotid or lower extremity artery stenosis;
ankle-brachial index <0.9; eGFR persistently <60 mL/min/1.73 m2; hypertension with
left ventricular hypertrophy; or persistent albuminuria
If age ≥60 years, any of the above or at least 2 of: any tobacco use; use of lipid-
modifying therapy or a documented untreated LDL cholesterol ≥3.4 mmol/L (130
mg/dL) within the past 6 months; HDL cholesterol <1.0 mmol/L (40 mg/dL) for men
and <1.3 mmol/L (50 mg/dL) for women or triglycerides ≥2.3 mmol/L (200 mg/dL)
within the past 6 months; use of ≥1 blood pressure drug or untreated systolic blood
pressure ≥140 mm Hg or diastolic blood pressure ≥95 mm Hg; or waist-to-hip ratio
>1.0 (men) and >0.8 (women)

The study is designed to consist of a screening visit followed by a single-blind 3-week placebo run-in period. Afterwards, patients are randomized to either dulaglutide 1.5 mg or placebo and followed at approximately 6-month intervals. Patients are followed until approximately 1200 patients experience a primary endpoint event, adjudicated as such.

The primary efficacy measure is time to first occurrence (after randomization) of the composite endpoint of death from CV causes, nonfatal myocardial infarction (MI), or nonfatal stroke. Secondary outcomes include each component of the primary composite cardiovascular outcome, a composite clinical microvascular outcome comprising retinal or renal disease, hospitalization for unstable angina, heart failure requiring hospitalization or an urgent heart failure visit, and all-cause mortality.

Exploratory analyses include assessment of the effects of dulaglutide on cognitive decline, as measured through administration of 2 different cognitive instruments—MoCA and DSST— at baseline and at the 2-year, 5-year and end-of-study visits. The MoCA and

DSST methods are described in more detail above.

The primary cognitive outcome is country-standardized substantive cognitive decline (SCD), which is defined as a reduction of either the MoCA or DSST score of ≥1.5 standard deviations below the baseline mean score in the participant's country. Country-standardization is accomplished by first calculating the baseline mean and standard deviation of the MoCA and DSST score within each country. These baseline mean and standard deviations are then used to calculate a standardized MoCA and DSST score for each participant at each time point. This is done by subtracting the country-specific baseline mean score from the individual's score at that time point and dividing the difference by the country specific baseline standard deviation. Additional cognitive outcomes included composites of SCD with death, stroke, stroke or TIA, and stroke, TIA or death, and the change in standardized MoCA and DSST scores over time.

These cognitive analyses are restricted to those participants who have a baseline MoCA or DSST score and at least 1 follow-up score of the same type.

Continuous variables are summarized using means with standard deviations and binary variables are summarized using counts with percentages. Cox proportional hazard models are used to estimate the hazard of SCD and SCD-based composite outcomes with dulaglutide versus placebo, both before and after accounting for each individual's baseline standardized MoCA and DSST scores. Sensitivity of the Cox model to the discrete nature of the results due to the intermittent administration of the cognitive tests is assessed by repeating the main analyses using a discrete time proportional odds logistic model. Where indicated, Cox models account for the competing risk of death. See Fine J P, Gray R J. A proportional hazards model for the sub distribution of a competing risk. Journal of the American Statistical Association 1999; 94: 496-509. Differences for effects within subgroups are explored by including the subgroup, and a subgroup-treatment interaction term in the model. Finally, repeated measures linear mixed-effects models estimate the effect of dulaglutide on the least square mean difference in the standardized MoCA and DSST scores, with the standardized baseline scores as covariates, participant as a random effect, and fixed effects for treatment, visit, and treatment—visit interaction. See Senn S. Change from baseline and analysis of covariance revisited. Stat Med 2006; 25(24): 4334-44.

All reported p-values are 2-sided and a nominal level of significance of 0.05 is used. All statistical analyses are done using SAS software (version 9.4).

12,137 individuals were screened, and 9901 individuals in 370 sites located in 24 countries were randomly allocated to either dulaglutide or placebo. The first participant was randomized in August 2011 and recruitment ended in August 2013Patients were and followed until August 2018. During a median follow-up of 5.4 years (interquartile range 5.1, 5.9) comprising 51,820 person-years, the final composite outcome status was known in 9610 patients.

Results were available for 8828 participants who had a baseline MoCA or DSST score and at least 1 follow-up score of the same type. Characteristics of those participants are provided in Table 2 below.

TABLE 2
Baseline Clinical Characteristics of Cognitive Study Participants.

| | All Participants | Dulaglutide | Placebo |
|---|---|---|---|
| Characteristic | N = 8828 | N = 4456 | N = 4372 |
| Age (years)-mean (SD) | 66 (6.4) | 66 (6.4) | 66 (6.4) |
| Females-N (%) | 4108 (46.5) | 2081 (46.7) | 2027 (46.4) |
| White-N (%) | 6742 (76.4) | 3421 (76.8) | 3321 (76.0) |
| Education ≤12 years-N (%) | 5350 (60.6) | 2717 (61.0) | 2633 (60.2) |
| Current tobacco use-N (%) | 1241 (14.1) | 616 (13.8) | 625 (14.3) |
| Cardiovascular disease-N (%)^{a} | 2724 (30.9) | 1373 (30.8) | 1351 (30.9) |
| Prior Stroke or TIA-N (%) | 781 (8.8) | 388 (8.7) | 393 (9.0) |
| Hypertension-N (%) | 8216 (93.1) | 4150 (93.1) | 4066 (93.0) |
| Atrial fibrillation-N (%) | 561 (6.4) | 293 (6.6) | 268 (6.1%) |
| Heart failure-N (%) | 755 (8.6) | 374 (8.4) | 381 (8.7) |
| Diabetes duration (y)-mean | 10.4 (7.1) | 10.3 (7.1) | 10.4 (7.1) |
| Diabetic retinopathy-N (%) | 801 (9.1) | 406 (9.1) | 395 (9.0) |
| Albuminuria^{b}-N (%) | 2992 (33.9) | 1491 (33.5) | 1501 (34.3) |
| Body Mass Index (kg/m²)- | 32.4 (5.7) | 32.4 (5.7) | 32.4 (5.7) |
| Systolic BP (mm Hg)-mean | 137.0 (16.7) | 136.9 (16.5) | 137.2 (17.0) |
| Diastolic BP (mm Hg)-mean | 78.5 (9.8) | 78.4 (9.8) | 78.6 (9.9) |
| HbA1c (%)-mean (SD) | 7.3 (1.1) | 7.3 (1.1) | 7.3 (1.0) |
| eGFR (ml/min/1.73 m²)-mean | 77.4 (22.5) | 77.7 (22.5) | 77.2 (22.6) |
| LDL cholesterol (mmol/L)- | 2.6 (1.0) | 2.6 (1.0) | 2.5 (1.0) |
| MoCA Mean (SD) | 24.6 (4.2) | 24.6 (4.1) | 24.6 (4.3) |
| MoCA Median (IQR) | 25.0 (22.0, 28.0) | 25.0 (22.0, | 25.0 (22.0, |
| Standardized MoCA | 0.0 (1.0) | −0.0 (1.0) | 0.0 (1.0) |
| Mean (SD) | | | |
| DSST Mean (SD) | 38.3 (19.6) | 38.0 (19.5) | 38.6 (19.8) |
| DSST Median (IQR) | 37.0 (25.0, 49.0) | 37.0 (25.0, | 37.0 (25.0, |
| Standardized DSST Mean | 0.0 (1.0) | −0.0 (1.0) | 0.0 (1.0) |
| (SD) | | | |

^{a}myocardial infarction, ischaemic stroke, unstable angina with electrocardiogram changes, myocardial Ischemia on imaging or stress test, or coronary, carotid or peripheral revascularization;
^{b}albumin/creatinine ≥3.39 mg/mmol.

The overall incidence of country-standardized SCD and analyses of the effect of dulaglutide in subgroups defined according to thirds of the baseline MoCA and DSST scores are shown below in Table 3. As seen in Table 3, the overall incidence of country-standardized SCD in people randomly assigned to dulaglutide and placebo was 4.05 and 4.34 per 100 person-years respectively (HR 0.93, 95% CI 0.85, 1.02; p=0.11). The effect of dulaglutide in subgroups defined according to thirds of the baseline MoCA and DSST score suggested a greater and statistically significant (P for heterogeneity 0.004 and 0.028 respectively) effect in people with the lowest baseline scores.

TABLE 3
Effect of dulaglutide on country-standardized substantive cognitive decline
for overall population and baseline score ranges defined according to the lowest,
middle and highest thirds of each defined by tertiles for each assay.

| | Dulaglutide | | Placebo | | | | |
|---|---|---|---|---|---|---|---|
| Measure | Events/Total | N/100py | Events/Total | N/100py | HR (95% CI) | P | P Int |
| SCD | 905/4456 | 4.05 | 944/4373 | 4.34 | 0.93 (0.85, 1.02) | 0.11 | |
| MoCA ≤ −0.282 | 589/1461 | 8.97 | 633/1449 | 9.99 | 0.89 (0.79, 0.99) | 0.034 | |
| MoCA > −0.282 | 217/1623 | 2.56 | 202/1476 | 2.63 | 0.98 (0.81, 1.18) | 0.80 | |
| to 0.606 | | | | | | | |
| MoCA > 0.606 | 91/1364 | 1.25 | 104/1441 | 1.35 | 0.93 (0.70, 1.23) | 0.59 | 0.004 |
| DSST ≤ −0.486 | 565/1501 | 8.26 | 600/1400 | 9.77 | 0.83 (0.74, 0.93) | 0.001 | |
| DSST −0.487 to | 219/1502 | 2.83 | 217/1485 | 2.84 | 1.00 (0.82, 1.20) | 0.96 | |
| 0.279 | | | | | | | |
| DSST > 0.279 | 106/1430 | 1.39 | 113/1468 | 1.44 | 0.96 (0.74, 1.25) | 0.77 | 0.028 |

Subsequent analyses were adjusted for each individual's baseline MoCA and DSST scores. Results are provided in Table 4 below.

TABLE 4

| Measure | HR (95% CI) | P |
|---|---|---|
| SCD adjusted for baseline scores | 0.86 (0.79, 0.95) | 0.002 |
| SCD adjusted for baseline scores and age | 0.86 (0.78, 0.94) | 0.001 |
| SCD adjusted for baseline scores, age, | 0.87 (0.79, 0.95) | 0.003 |
| ethnicity, education | | |
| SCD adjusted for baseline scores and | 0.87 (0.79, 0.96) | 0.003 |
| competing risk of death | | |
| Composite of SCD or death adjusted for | 0.88 (0.81, 0.95) | 0.001 |
| baseline scores | | |
| Composite of SCD or stroke adjusted for | 0.85 (0.77, 0.92) | <0.001 |
| baseline scores | | |
| Composite of SCD, stroke or TIA adjusted for | 0.85 (0.78, 0.92) | <0.001 |
| baseline scores | | |
| Composite of SCD, stroke, TIA or death | 0.87 (0.80, 0.94) | <0.001 |
| adjusted for baseline scores | | |

Abbreviations:
SCD = substantive cognitive decline;
HR = hazard ratio;
TIA = transient ischemic attack.

As noted in Table 4, after this adjustment, people in the dulaglutide group had a 14% lower hazard of SCD than those in the placebo group (HR 0.86, 95% CI 0.79, 0.95; p=0.002). Similar estimates of effect were noted after additional adjustment for age, education, ethnicity or the competing risk of death, and after including SCD within various composite outcomes.

Further, similar beneficial effects were also noted for people in the dulaglutide group compared to the placebo group across subgroups defined on the basis of age (≤70 years), education (≤12 years), prior cardiovascular disease, a prior stroke or TIA, standardized baseline MoCA score (≤1 unit), HbA1c (≤0.081%), the natural logarithm of the albumin:creatinine ratio (≤−0.062; −0.062 to 1.35; and >1.35) and eGFR (<60 mL/min/1.73m2). (P interaction>0.07 for all).

1. A method of treating, preventing or delaying development of a cognitive disorder in a patient, comprising administering dulaglutide in a therapeutically effective amount to the patient once weekly.

2. The method of claim 1, wherein the cognitive disorder is selected from the group consisting of MCI and dementia.

3. A method of preventing or delaying cognitive decline in a patient, comprising administering dulaglutide in a therapeutically effective amount to the patient once weekly.

4. The method of claim 1 wherein the patient has type 2 diabetes mellitus.

5. A method of improving glycemic control and preventing or delaying cognitive decline in a patient in a patient with type 2 diabetes mellitus, comprising administering dulaglutide in a therapeutically effective amount to the patient once weekly.

6. The method of claim 4, wherein the method results in a reduction in the risk of the patient experiencing cognitive decline.

7. A method of improving glycemic control in a patient with type 2 diabetes mellitus, comprising administering dulaglutide in a therapeutically effective amount to the patient once weekly, wherein the method results in a reduction in the risk of the patient experiencing cognitive decline.

8. The method of claim 4 wherein the patient's risk of cognitive decline is reduced by about 14%.

9. The method of claim 4, wherein the patient's risk of the occurrence of a composite of the following outcomes is reduced: cognitive decline, stroke, transient ischemic attack or death.

10. The method of claim 4 wherein the therapeutically effective amount of dulaglutide is selected from the group consisting of about 1.5 mg, about 3.0 mg and about 4.5 mg.

11. The method of claim 10 wherein the therapeutically effective amount of dulaglutide is about 1.5 mg.

12. The method of claim 4 wherein once weekly administration of dulaglutide is continued for at least 2 years.

13. The method of claim 4 wherein the patient has either: multiple cardiovascular risk factors without established cardiovascular disease; or established cardiovascular disease.
